(19) United States
(12) Patent Application Publication
(10) Pub. No.: US 2014/0275996 A1
(43) Pub. Date: Sep. 18, 2014

(54) SYSTEMS AND METHODS FOR CONSTRUCTING AN IMAGE OF A BODY STRUCTURE

(71) Applicant: VOLCANO CORPORATION, San Diego, CA (US)

(72) Inventor: Jeremy Stigall, Carlsbad, CA (US)

(73) Assignee: VOLCANO CORPORATION, San Diego, CA (US)

(21) Appl. No.: 14/204,696

(22) Filed: Mar. 11, 2014

Related U.S. Application Data
(60) Provisional application No. 61/777,860, filed on Mar. 12, 2013.

(51) Int. Cl. A61B 6/00; A61B 19/00; A61B 5/00; A61B 1/00; A61B 1/05; A61B 6/12; A61B 8/12

(52) U.S. Cl. CPC A61B 6/5247; A61B 6/12; A61B 19/54; A61B 6/504; A61B 8/12; A61B 1/00165; A61B 1/051; A61B 5/0066; A61B 5/0084; A61B 6/461
USPC 600/424

(57) ABSTRACT

The invention generally relates to systems and methods for constructing an image of a body structure. In certain embodiments, methods of the invention involve externally imaging a body structure within the patient using a first imaging device. The methods also involve internally imaging the body structure within the patient using a second imaging device. The second imaging device includes a radiopaque label co-located with an image collector of the second imaging device. Additionally, methods of the invention involve combining external imaging data and internal imaging data to produce an image of the body structure. The label on the second imaging device facilitates alignment of the external imaging data and the internal imaging data.

### RELATED APPLICATION

The present application claims the benefit of and priority to U.S. provisional patent application Ser. No. 61/777,860, filed Mar. 12, 2013, the content of which is incorporated by reference herein in its entirety.

### FIELD OF THE INVENTION

The invention generally relates to systems and methods for constructing an image of a body structure.

### BACKGROUND

Atherosclerosis is treated in arteries of the heart, head, neck and peripheral portions of the body using many different methods. The most popular methods, such as angioplasty, bare metal stenting, drug eluting stenting (permanently implantable and biodegradable), various types of energy delivery and rotational atherectomy, all treat an artery equally around the circumference of a target length of the arterial lumen. These devices are generally circumferentially symmetric, and cannot selectively treat one circumferential sector of the targeted length of the artery any different from another. Almost always, the targeted length of the artery identified for treatment is determined using angiography, which graphically depicts a vessel lumen, or intravascular ultrasound (IVUS), which graphically depicts the atherosclerotic plaque itself. With IVUS, the thickness of the atherosclerotic plaque can be determined along the length of the diseased area and at specific radial positions around its circumference. More often than not, the plaque is eccentric and thus varies in thickness at particular positions of a circumferential cross-sectional of the vessel. Treatment of plaque using the aforementioned circumferentially symmetric methods can sometimes cause undesired results. For example, drug eluting stents deliver drugs that inhibit neo-intimal proliferation (known as restenosis). In the section of artery where the stent is expanded, any normal (non-diseased) portion of vessel may not benefit from getting the same dosage of drug as the diseased portion.

Some methods for treating atherosclerosis, such as directional athrectomy, needle aided drug injection or certain types of brachytherapy (radiation), can actually vary the treatment along different circumferential sectors of the artery. The catheters used for these treatment methods are typically circumferentially asymmetric and have at least a portion that is torquable (rotatable), and thus able to be steered into a desired circumferential orientation. However, effective use of the asymmetric treatments is difficult because of certain characteristics of current imaging methods. For example, because angiography only shows an image of the lumen of the blood vessel, it is impossible to identify exactly where, in a particular circumferential cross-section, the atherosclerotic plaque is located and the plaque's thickness. IVUS does make it possible to view the circumferential location and thickness of atherosclerotic plaque in a length of a vessel, but unless the ultrasonic transducer is attached to the actual treatment device, it is difficult to use the IVUS image to direct the treatment catheter with precision. This is especially difficult in coronary arteries, where heart motion adds error. Attempts to include transducers on the treatment catheter have been moderately successful (U.S. Pat. No. 6,375,615 to Flaherty) but the additional components make it more difficult to build a small catheter, which is flexible and can track easily in the artery. Some other catheters have been developed (U.S. Pat. Nos. 4,821,731 and 5,592,939, both to Martinelli) which can combine IVUS imaging with tip positioning technology. This enables displaying a three dimensional graphical representation of the plaque, including any tortuosity inherent in the artery. However, additional capital equipment is required in the procedure room to perform this type of imaging and adds cost to performing the procedure.

### SUMMARY

The invention provides imaging catheters, methods, and systems that will benefit both the patient and technician/physician by making the precise location of an intravascular image easier to identify in an accompanying angiogram. By co-locating a radiopaque label with the image collector of an imaging catheter, it is easier to identify the exact location of the image collector and to correlate a given image with a specific location within the vasculature. The improvement in the image collector makes possible systems that can simultaneously display an intravascular image and pinpoint the location of that image on a corresponding angiogram.

In one aspect, the invention is an imaging catheter including a radiopaque label co-located with the image collector. The image collector can be a piezoelectric sensor, a micromachined transducer, a photodiode, a charge coupled device, a microchannel array, a lens, or an optical fiber. The catheter can be used to collect intravascular ultrasound (IVUS), intravascular optical coherence tomography (OCT), intravascular Doppler, or intravascular visible images. Because the radiopaque label does not transmit medical x-rays, it shows up as a dark spot in a fluoroscopic image of the subject, allowing a physician to quickly identify the location of an intravascular image obtained with the collector.

In another aspect, the invention is a method for locating the position of an intravascular image in a subject. The method includes inserting an intravenous imaging catheter having a radiopaque label co-located with an image collector into a subject and imaging a portion of the vasculature of the subject using the image collector. During or after the imaging, the area of the body of the patient where the catheter is located is imaged to determine the precise location of the radiopaque label and thus the location of the intravascular image is also known.

In another aspect, the invention is a system for locating the position of an intravascular image in a subject. The system includes a processor and a computer readable storage medium having instructions that when executed cause the processor to execute the methods of the invention. For example, the instructions may cause the processor to receive imaging data of vasculature of a subject collected with an image collector co-located with a radiopaque label and then subsequently receive an image (e.g., angiogram) of the subject including the radiopaque label. Once the radiopaque label has been located in the image of the subject, the system outputs an image of the subject showing the location of the image collector and outputs an intravascular image of the vasculature of a subject. In some instances, the processor will output an image that simultaneously shows the location of the image collector and the vasculature of the subject. The system may additionally include the tools needed to obtain and process the imaging data and images, such as catheters, fluoroscopes, and related control equipment.

In certain embodiments, creating, in a coordinated manner, graphical images of a body including vascular features from a combination of image data sources, in accordance with the present invention, includes initially creating an external ultrasound image of a vessel segment. The external ultrasound image is, for example, either a two or three dimensional image representation. Next, a vessel image data set is acquired that is distinct from the external ultrasound image data. The vessel image data set includes information acquired at a series of positions along the vessel segment. An example of such vessel image data is a set of intravascular ultrasound frames corresponding to circumferential cross-section slices taken at various positions along the vessel segment. The external ultrasound image and the vessel image data set are correlated by comparing a characteristic rendered independently from both the external ultrasound image and the vessel image data at positions along the vessel segment.

### BRIEF DESCRIPTION OF THE DRAWINGS

FIG. 1 is a graphical illustration of a three dimensional length of artery, including a highly diseased segment.

FIG. 2 is a graphical illustration of a portion of the artery depicted in FIG. 1 with a longitudinal section removed along lines 2 to illustratively depict different elements of atherosclerotic plaque.

FIG. 3 is a graphical illustration of the artery from FIGS. 1 and 2 wherein an imaging catheter has been inserted in the artery.

FIG. 4 is detailed view of a section of the artery depicted in FIG. 3 including an imaging catheter in the artery.

FIG. 5A shows a graphical display interfaces rendered by a tissue characterization system for use with intravascular ultrasound (IVUS). FIG. 5B shows a graphical display interfaces rendered by a tissue characterization system for use with intravascular ultrasound (IVUS).

FIG. 6A is a set of graphical images depicting a three-dimensional reconstruction method using two two-dimensional angiographic images.

FIG. 6B is a flowchart depicting a set of exemplary steps for creating a co-registered three-dimensional graphical display.

FIG. 7 illustratively depicts a vessel reconstruction graphical image based on image creation techniques embodied in a system and method incorporating the present invention.

FIG. 8A illustratively depicts the use of a directional atherectomy catheter according to guidance provided from the vessel reconstruction. FIG. 8B illustratively depicts the use of a directional atherectomy catheter according to guidance provided from the vessel reconstruction. FIG. 8C illustratively depicts the use of a directional atherectomy catheter according to guidance provided from the vessel reconstruction.

FIG. 9 illustratively depicts a series of custom, single link stents which have been crimped onto a dilatation balloon for placement in a diseased blood vessel.

FIG. 10 illustratively depicts a graphical display image including an overlay of the reconstruction over a live two-dimensional angiographic image.

FIG. 11 illustratively depicts a first graphic display in relation with a graphical representation of a three-dimensional or two-dimensional image.

FIG. 12 illustratively depicts a second graphic display in relation with a graphical representation of a three-dimensional or two dimensional image.

FIG. 13 illustratively depicts a third graphic display in relation with a graphical representation of a three-dimensional or two-dimensional image.

FIG. 14 illustratively depicts a graph including two separate sequences of values corresponding to lumen area, in relation to image frame number and linear displacement along an imaged vessel, prior to axial registration adjustment.

FIG. 15 illustratively depicts a graph including two separate sequences of values corresponding to lumen area, in relation to image frame number and linear displacement along an imaged vessel, after axial registration adjustment.

FIG. 16 illustratively depicts a graphical display of angiography and vessel (e.g., IVUS) images on a single graphical display prior to axial registration adjustment.

FIG. 17 illustratively depicts a graphical display of angiography and vessel (e.g., IVUS) images superimposed on a single graphical display after axial registration adjustment.

FIG. 18 illustratively depicts the process of circumferential registration of angiography and vessel (e.g., IVUS) image sets.

FIG. 19 illustratively depicts angular image displacement in relation to circumferential registration of angiography and vessel (e.g., IVUS) image sets.

FIG. 20 illustratively depicts a graph of actual and best fit rotational angle corrections displayed in relation to image frame number.

FIG. 21 shows a catheter for taking intravascular images having a radiopaque label co-located with the image collector.

FIG. 22 shows the detail of the collector assembly, including a radiopaque label co-located with the image collector;

FIG. 23 is a simultaneous display of an intravascular ultrasound (IVUS) image and an angiogram of the artery from which the IVUS image originated;

FIG. 24 is an alternative simultaneous display of an IVUS image and an angiogram of the artery from which the IVUS image originated;

FIG. 25 is a simultaneous display of an optical coherence tomography (OCT) image and an angiogram of the artery from which the OCT image originated;

FIG. 26 is a flowchart of a system of the invention;

FIG. 27 is block diagram of a system of the invention for locating the position of an intravascular image relative to an image of the vasculature of the subject;

FIG. 28 is a block diagram of a networked system for locating the position of an intravascular image relative to an image of the vasculature of the subject.

### DETAILED DESCRIPTION

In FIG. 1, a diseased artery 5 with a lumen 10 is shown. Blood flows through the artery 5 in a direction indicated by arrow 15 from proximal end 25 to distal end 30. A stenotic area 20 is seen in the artery 5. FIG. 2 shows a sectioned portion of the stenotic area 20 of the artery 5. An artery wall 35 consists of three layers, an intima 40, a media 45 and an adventitia 55. An external elastic lamina (EEL) 50 is the division between the media 45 and the adventitia 55. A stenosis 60 is located in the artery 5 and limits blood flow through the artery. A flap 65 is shown at a high stress area 70 of the artery 5. Proximal to the stenosis 60 is an area of vulnerability 75, including a necrotic core 80. A rupture commonly occurs in an area such as the area of vulnerability 75.

FIG. 3 illustratively depicts an imaging catheter 85 having a distal end 95 that is inserted into the stenotic area 20 of the artery 5. The imaging catheter 85 is inserted over a guidewire 90, which allows the imaging catheter 85 to be steered to the desired location in the artery 5. As depicted in FIG. 4, the imaging catheter 85 includes an imaging element 100 for imaging the diseased portions and normal portions of the artery 5. The imaging element 100 is, for example, a rotating ultrasound transducer, an array of ultrasound transducer elements such as phased array/cMUT, an optical coherence tomography element, infrared, near infrared, Raman spectroscopy, magnetic resonance (MRI), angioscopy or other type of imaging technology. Distal to the imaging element 100 is a tapered tip 105 which allows the imaging catheter 85 to easily track over the guidewire 90, especially in challenging tortuous, stenotic or occluded vessels. The imaging catheter 85 can be pulled back or inserted over a desired length of the vessel, obtaining imaging information along this desired length, and thereafter creating a volumetric model of the vessel wall, including the diseased and normal portions, from a set of circumferential cross-section images obtained from the imaging information. Some technologies, such as IVUS, allow for the imaging of flowing blood and thrombus.

FIGS. 5A and 5B illustratively depict, by way of example, features of a vascular tissue characterization system marketed by Volcano Corporation. As graphically depicted in angiogram 130, a two dimensional image of an artery lumen 135 on its own does not provide visual information about atherosclerotic plaque that is attached to walls of an artery containing the lumen 135. Instead the angiogram 130 only depicts information about a diameter/size of the lumen 135 through which blood flows. A Gray scale IVUS cross-sectional image 115 demonstrates a cross-sectional view of the lumen 135 and atherosclerotic plaque that surrounds the lumen. Known automatic border detection algorithms executed by an IVUS image data processing system facilitate identifying a luminal boundary 125 and an EEL 110. Plaque components are identified from information derived from IVUS radiofrequency backscatter and are color coded. The various characterized and graphically depicted plaque components potentially consist, by way of example, fibrous, fibro-lipidic (fibro fatty), necrotic core, calcified (dense calcium), blood, fresh thrombus, and mature thrombus. The RF backscatter can also give information to identify and color code stent materials such as metallic or polymeric stents. The distribution of components in a cross-section or in the entire volume of the vessel analyzed is displayed by way of example through various graphics depicted in a bracketed portion 145 of an exemplary graphical display depicted in FIG. 5B. In addition to the cross-sectional display images rendered in portion 145, a longitudinal display region 140 is also included in the illustrative graphical display in FIG. 5B that depicts information obtained from portions of a set of circumferential cross-sectional slides.

FIG. 6A illustratively depicts the general concept behind a prior art three-dimensional reconstruction analysis system. A first two-dimensional angiographic image 150 taken in a first view plane and a second two-dimensional angiographic image 155, taken in a second view plane differing from the first view plane are combined and analyzed to create a graphical representation of a three-dimensional image depicted on a graphical display 160. The image displayed on the graphical display 160 provides a much more realistic graphical representation of a lumen of an actual artery (or other blood vessel) than the typical two-dimensional angiography images.

In accordance with an aspect of an imaging system embodying the present invention, IVUS images are co-registered with the three-dimensional image depicted on the graphical display 160. Fiduciary points are selected when the imaging catheter is at one or more locations, and by combining this information with pullback speed information, a location vs. time (or circumferential cross-sectional image slice) path is determined for the imaging probe mounted upon the catheter. Co-registering cross-sectional IVUS with three-dimensional images of the type depicted in FIG. 6a allows for a three-dimensional volumetric map of either gray scale images or colorized tissue characterization (tissue composition) images.

Turning to FIG. 6B, a set of steps are depicted for creating a volumetric map. The particular order of the steps differs in alternative embodiments. During step 162, the imaging catheter is pulled back either manually or automatically through a blood vessel segment, and a sequence of circumferential cross-sectional IVUS image frames is acquired/created. During step 163 an angiographic image is formed of the blood vessel segment. The image is, for example, a two-dimensional image or, alternatively a three-dimensional image created from two or more angiographic views. During step 164, at least one fiduciary point is designated on the angiographic image, either by the user, or automatically by the imaging system. During step 166, the angiographic image and the information obtained from the imaging catheter during the pullback are aligned/correlated using the fiduciary point locating information. Thereafter, during step 168 the cross-sectional IVUS images are displayed on a graphical display in association with a two- or three-dimensional graphical representation of the imaged vessel. The graphical representation of the imaged vessel is based at least in-part upon the angiographic image information. By way of example, in an exemplary embodiment, the angiographic image itself is displayed. In an alternative embodiment, information from an angiographic image is only used to guide piece-wise reconstruction of the imaged vessel from the sequence of IVUS image slices by determining the linear displacement and orientation of adjacent sections of the reconstructed vessel using the angiographic image of the vessel.

Turning to FIG. 7, by combining or overlaying the three-dimensional map of imaging information over the three-dimensional image 160 of the vessel lumen, or over one or more two-dimensional views of the angiogram, a reconstruction 165 that more realistically represents the actual vessel is obtained, which is correct in its portrayal of vessel tortuosity, plaque composition and associated location and distribution in three dimensions. For example, a necrotic core which is located in the vessel in the sector between 30° to 90°, also having a certain amount of longitudinal depth, will appear on the reconstruction 165 with the same geometry. An augmented overall vessel diameter, due to thickened plaque, will also appear this way in the reconstruction 165. The additional information from the non-angiography imaging data makes displaying such vessel images possible. The steps of the procedure summarized in FIG. 6A facilitate co-registration of the IVUS information over a live two-dimensional angiographic image, giving the operator the ability to view a projection of the volume of plaque over a two-dimensional image of the lumen. The co-registered displayed graphical image allows an operator to make a more informed diagnosis, and also allows the operator to proceed with therapeutic intervention with the additional information provided by the co-registered displayed image guiding the intervention.

In the case of live two-dimensional or three-dimensional co-registration, one or more fiduciary points are selected first, followed by alignment by the system, and then simultaneous pullback and angiography or fluoroscopy. Note that in both co-registration in playback mode and co-registration in “live” mode, the information used by the system includes both the specific pullback speed being used (for example 0.5 millimeters per second) and the time vector of the individual image frames (for example IVUS image frames). This information tells the system where exactly the imaging element is located longitudinally when the image frame is (or was) acquired, and allows for the creation of an accurate longitudinal map.

Automatic fiduciary points are used, for example, and are automatically selected by the system in any one of multiple potential methods. A radiopaque marker on the catheter, approximating the location of the imaging element, for example is identified by the angiography system, creating the fiduciary point. Alternatively, the catheter has an electrode, which is identified by three orthogonal pairs of external sensors whose relative locations are known. By measuring field strength of an electrical field generated by the probe, the location of the electrode is “triangulated”.

FIG. 7 graphically depicts a reconstruction produced using the techniques discussed above. Three necrotic cores 80a, 80b and 80c have been identified. First necrotic core 80a is located at twelve o'clock circumferentially in the vessel and is identified as being located in the stenosis 60, and deep beneath a thickened cap. The location of the necrotic core 80a beneath the thickened cap suggests that this necrotic core is more stable than the other two necrotic cores—core 80b which is very close to the surface, and 80c which is also close to the surface. As shown in this reconstruction, and in relation to the first necrotic core 80a, the second necrotic core 80b is located at nine o'clock and the third necrotic core 80c is circumferentially located at four o'clock. This circumferential information is employed, for example, to localize application of appropriate treatment. The graphically depicted information provided by the imaging catheter and reconstruction allows delivery of the therapeutic catheter to the precise treatment location, with the desired catheter orientation. Alternatively, the imaging catheter itself is a combination imaging and therapy catheter, and the treatment simultaneously coincides with the imaging. One possible treatment scenario involves placing a drug eluting stent at the portion of the depicted vessel near the stenosis 60 and treating the second and third necrotic cores 80b and 80c by a needle-based drug, cell (i.e. stem cell) or gene delivery catheter (U.S. Pat. No. 6,860,867 to Seward), or by removing the necrotic core material by a needle and vacuum catheter. If using a tissue removal technique, such as atherectomy, ultrasonic therapeutics, or a plaque modification technique such as photodynamic therapy, drug delivery, radiation, cryoplasty, radiofrequency heating, microwave heating or other types of heating, the knowledge of the location of the EEL 50 is important. This assures that the adventitia is not disturbed, and that vessel perforation does not occur. The reconstruction 165 is graphically displayed in a manner that clearly demonstrates the location of the EEL 50 from all viewing angles. It can be seen that the thickness 170 between the luminal boundary and the EEL 50 at the stenosis 60 is much larger than the thickness 175 between the luminal boundary and the EEL 50 proximal to the stenosis 60. The circumferential (azimuthal) and radial (depth) orientation of the plaque components has been discussed herein above, but the axial (longitudinal) orientation/positioning—the distances separating diseased sections along a vessel's length—is important also. First necrotic core 80a is further distal than second necrotic core 80b, and second necrotic core 80b is further distal than third necrotic core 80c. The axial arrangement (lengthwise positioning) of diseased sections is important when choosing a particular length of a stent to use, or where to place the distal-most or proximal-most portion of the stent. It is also important when determining the order or operation in the treatment sequence. In addition, very proximally located vulnerable plaques are generally of greater concern than distally located vulnerable plaques, because they supply blood to a larger volume of myocardium.

Arteries also have side branches which can be identified with imaging techniques such as standard IVUS imaging, or IVUS flow imaging (which identifies the dynamic element of blood). The side branches are potentially used as fiduciary points for axial, circumferential and even radial orientation of the IVUS information, with respect to an angiographic base image, which also contains side branch information.

Turning to FIGS. 14-20, an exemplary technique is illustrated for obtaining accurate axial and circumferential co-registration of IVUS information (or other image information obtained via a probe inserted within a body) with the three-dimensional image 160. Turning initially to FIG. 14 and FIG. 15, the illustrations are intended to represent the internal representation of information created/processed by the imaging/display system. However, in an illustrative embodiment, such information is presented as well as graphical displays rendered by the system, in the manner depicted in FIGS. 14 and 15 as a visual aid to users in a semi-automated environment. For example, a user can manually move the relative positioning of a sequence of IVUS frames with regard to linear displacement of a vessel as depicted in corresponding data values generated from an angiographic image.

Furthermore, as those skilled in the art will readily appreciate, the line graphs in FIGS. 14 and 15 corresponding to IVUS frames comprise a sequentially ordered set of discrete values corresponding to a sequence of “N” frames of interest. Similarly, values generated from angiographic image data are also taken at discrete points along a length of a vessel of interest. Thus, while depicted as continuous lines in the drawing figures, the values calculated from angiographic and IVUS information correspond to discrete points along the length of the vessel.

FIG. 14 includes a graph 320 depicting calculated/estimated lumen area as a function of IVUS image frame number for both angiography and IVUS. The graph depicted in FIG. 14 shows the effect of inaccurate co-registration between two imaging methods and associated measured parameters (e.g., lumen cross-section size). A line graph 330 representing lumen area calculated from IVUS information and a line graph 325 representing lumen area calculated from angiography information are shown in an exemplary case wherein the measurements are misaligned along a portion of a vessel.

FIG. 14 corresponds to a graphically displayed composite image depicted in FIG. 16 that includes a graphical representation of a three-dimensional angiographic image 335 and a graphical representation of corresponding IVUS information 340 where the two graphical representations are shifted by a distance (“D”) in a composite displayed image. The misalignment is especially evident because minimum luminal circumferential cross-section regions (i.e., the portion of the vessel having the smallest cross-section) in the images graphically rendered from each of the two data sets do not line up. The minimal lumen area calculated from the IVUS information at point 345 in FIG. 14 corresponds to the IVUS minimal lumen position 360 in FIG. 16. The lumen area calculated from the angiography information at point 350 in FIG. 14 corresponds to the angiography minimal lumen position 355 in FIG. 16. Note that in the illustrative example, thickness of the vessel wall is depicted as substantially uniform on IVUS. Thus, an IVUS image frame where the minimal lumen area occurs is also where the minimum vessel diameter exists. This image feature differs from restricted flow due to a blockage within a diseased artery such as the one depicted in FIG. 2.

A lumen border 380 is also shown in FIG. 16. In order to achieve axial alignment between the graphical representation of the three-dimensional angiographic image 335 and the graphical representation of corresponding IVUS information 340, an axial translation algorithm is obtained based upon a “best-fit” approach that minimizes the sum of the squared differences between luminal areas calculated using the angiographic and the IVUS image data.

The best axial fit for establishing co-registration between angiogram and IVUS data is obtained where the following function is a minimum.

$${\sum }_{n = 1}^{N} {( {A}_{Lumen} - {A}_{Angio} )}^{2} ;$$

with A_{Lumen}=IVUS lumen area for frames n=1, N and A_{Angio} angiography area for “frames” n=1, N (sections 1-N along the length of an angiographic image of a blood vessel). By modifying how particular portions of the angiographic image are selected, the best fit algorithm can perform both “skewing” (shifting all slices a same distance) and “warping” (modifying distances between adjacent samples).

Using the axial alignment of frames where the summation function is a minimum, a desired best fit is obtained. FIGS. 15 and 17 depict a result achieved by realignment of line graphs and corresponding graphical representations generated from the angiographic and IVUS data, depicted in a pre-aligned state in FIGS. 14 and 16, based upon application of a “best fit” operation on frames of IVUS image data and segments of a corresponding angiographic image,

FIG. 18 illustratively depicts a graphical representation of a three-dimensional lumen border 365 rendered from a sequence of IVUS image slices after axially aligning a three-dimensional angiographic data-based image with a graphical image generated from IVUS information for a particular image slice. The displayed graphical representation of a three dimensional image corresponds to the lumen border 380 shown in FIG. 17. The lumen border 380 is shown projected over a three-dimensional center line 385 obtained from the angiographic information. FIG. 18 also depicts a first angiography image plane 370 and a second angiography image plane 375 that are used to construct the three dimensional center line 385 and three-dimensional angiographic image 335. Such three-dimensional reconstruction is accomplished in any one of a variety of currently known methods. In order to optimize the circumferential orientation of each IVUS frame, an IVUS frame 400 depicting a luminal border is projected against the first angiography plane 370, where it is compared to a first two-dimensional angiographic projection 390. In addition, or alternatively, the IVUS frame 400 is projected against the second angiography image plane 375, where it is compared to the second two-dimensional angiographic projection 395 for fit. Such comparisons are carried out in any of a variety of ways including: human observation as well as automated methods for comparing lumen cross section images (e.g., maximizing overlap between IVUS and angiogram-based cross-sections of a vessel's lumen).

Positioning an IVUS frame on a proper segment of a graphical representation of a three-dimensional angiographic image also involves ensuring proper circumferential (rotational) alignment of IVUS slices and corresponding sections of an angiographic image. Turning to FIG. 19, after determining a best axial alignment between an IVUS image frame, such as frame 400, and a corresponding section of a three-dimensional angiographic image, the IVUS frame 400 is then rotated in the model by an angular displacement 405 (for example 1.degree.), and the fit against the angiographic projections is recalculated. As mentioned above, either human or automated comparisons are potentially used to determine the angular displacement. After this has been done over a range of angular orientations, the best fit angular rotation is determined.

FIG. 20 depicts a graph 410 of best angle fit and frame number. During the pullback of the IVUS catheter, there may be some slight rotation of the catheter, in relation to the centerline of the blood vessel, and so, calculating the best angular fit for one IVUS frame does not necessarily calculate the best fit for all frames. The best angular fit is done for several or all frames in order to create the graph 410 including actual line 412 and fit line 414. The actual line 412 comprises a set of raw angular rotation values when comparing IVUS and angiographic circumferential cross-section images. The fit line 414 is rendered by applying a limit on the amount of angular rotation differences between adjacent frame slices (taking into consideration the physical constraints of the catheter upon which the IVUS imaging probe is mounted). By way of example, when generating the fit line 414, the amount of twisting between frames is constrained by fitting a spline or a cubic polynomial to the plot on the actual line 412 in graph 410.

Having described an illustrative way to co-register angiographic and IVUS images for graphically representing a three-dimensional image of a vessel, attention is directed to FIGS. 8A, 8B and 8C that demonstrate the use of a directional atherectomy catheter 180 using guidance from the reconstruction 165. The directional atherectomy catheter 180 has a tapered receptacle tip 190 and a cutter window 185. In use, the catheter is manipulated, using a balloon or an articulation, to force the cutter window 185 against the atherosclerotic plaque so that the plaque protrudes into the cutter window 185. The plaque is then sliced off by a cutter (not shown) and collected in the tapered receptacle tip 190. In order to debulk the artery as much as possible (remove the plaque) it is desirable to cut away the plaque up to, but not past the EEL 50. The reconstruction 165 is used as a guide to track the directional atherectomy catheter 180 into a desired axial location along the length of the vessel. Thereafter, the catheter 180 is “torqued” (rotated at least partially) until the cutter window 185 is in the desired circumferential orientation. One in position the balloon or articulation is activated until the cutter window 185 is set up to allow the cutting of desired plaque but not adventitial tissue. In other words, only tissue within the EEL 50 boundary is excised.

With continued reference to FIG. 8A, using the reconstructed co-registered angiographic and IVUS images as a guide for the procedure, the directional atherectomy catheter 180 is tracked into place and torqued opposite an upper portion 60a of the stenosis. In FIG. 8B, an appropriate catheter mechanism, such as a balloon (not shown) is activated to force the cutting window 185 against an upper portion 60a of the stenosis. The upper portion 60a of the stenosis is then excised. During this cutting operation, the reconstruction procedure that achieves co-registration of the angiographic and IVUS images on a graphical three-dimensional rendering of a vessel allows the user to be fully aware of the location of the EEL 50, and thus the user knows when to stop articulating and cutting. FIG. 8C shows a directional atherectomy catheter 180 being tracked, torqued and articulated so that it can cut a lower portion 60b of the stenosis, again using a co-registered IVUS cross-sectional image to avoid cutting past the EEL 50 and into the adventitia 55 [Blair, need to add 55 to the drawing 8C. This is especially useful in debulking areas of large plaque volume, such as in the arteries of the leg (femoral, popliteal). The debulking is performed using the vessel visualization apparatus and methods described herein that are based upon use of both angiographic and IVUS image data. Debulking or other therapies may also be done using this smart visualization, and in combination with automated or semi-automated robotic or magnetic catheter manipulation systems.

Turning to FIG. 9, a dilatation balloon catheter 195 is prepared based on information derived from the reconstruction 165 of FIG. 7. A first stent 200a, second stent 200b and third stent 200c are crimped onto the dilatation balloon catheter 195 or attached by other methods known in the art. The first stent 200a is configured to correspond with stenosis 60. The stent is made from a mesh that has a higher metal to artery ratio than the other stents, to prevent distal embolization from unorganized thrombus which may occur near the flap 65. The stent may or may not be drug eluting. For example, if the artery is 3.5 mm or larger, a drug eluting stent is not always necessary to prevent restenosis. However, most fiberatheromas will necessitate a drug eluting stent to prevent in-stent restenosis. Because the first necrotic core 80a is deep within the stenosis, the stent serves more as a mechanical support for the entire dilated stenosis, rather than protection against rupture of this portion of the blood vessel. In contrast, necrotic cores 80b and 80c are closer to the lumen of the vessel and need to be treated in a more urgent manner. Second stent 200b is configured to be expanded over the second necrotic core 80b. A biodegradable stent (such as magnesium or a polymeric material) may be chosen, because it will be expanded in an area that does not require a high radial force to keep the artery open (this is already a non-stenotic area). The stent is designed to elute a statin, and the statin is more heavily dosed at the nine o'clock portion (not shown in FIG. 9) that corresponds with the second necrotic core 80b. The third stent 200c is the same as the second stent, 200b, except that it is oriented on the catheter with the more heavily dosed area 230 at four o'clock, in order to correspond with the third necrotic core 80c. By more properly dosing the drugs on the stents, there is less risk of wasted drug from high doses, being leaked systemically into a patient's body, and potentially causing harmful side effects. Not shown in this figure is another stent configuration that has a side hole that allows the stent to be placed over a sidebranch without obstructing flow of blood to the sidebranch. The image co-registration reconstruction method and apparatus described herein is also capable of identifying the size, location and orientation of sidebranches, and can be used to orient (circumferentially, axially) a sidehole stent of this design.

The catheter in FIG. 9 has four radiopaque markers 205a-205d, which delineate the positions of the three different short stents 200a, 200b and 200c. The catheter also has radiopaque markings or stripes that allow its circumferential orientation to be visible on X-ray. For example, a radiopaque marker band that does not completely encircle the catheter, so that visible portions and non-visible portions can be identified around the circumference of the marker.

FIG. 10 shows an overlay 235 of the reconstruction 165 placed over the live anigiography image 240. As a catheter is tracked through the vessel, the atherosclerotic plaque 225 and the EEL 220 is identified. In combination with tissue characterization and colorization, structures of concern 245 are easily identified in relation to the live image 240. Sidebranches 250 are used, for example, to align and co-register the two different images. Combining the three-dimensional reconstruction with tissue characterization information and a live two-dimensional angiography image, facilitate tracking and manipulating a therapeutic catheter (not shown) to areas that are of primary concern. It also allows for a more informed awareness of the state of vulnerability of various regions of the vessel. In the stenosis, target plaque 255 is viewed against a live two-dimensional angiography image to better aid plaque removal techniques, such as directional atherectomy.

FIGS. 11, 12 and 13 illustratively depict three different graphic displays for graphically representing information relating to plaque size and composition. A vessel lumen trace 260 is, for example, either a three-dimensional rendering of the vessel lumen (for example derived from two two-dimensional angiography images) or a two-dimensional projection of the three dimensional rendering. Alternatively, vessel lumen trace 260 is represented by a live angiographic image. In all of the aforementioned alternative angiographic imaging modes, it is possible to overlay images of the atherosclerotic plaque, however, it is difficult to appreciate the thickness, contours and composition of the plaque at all points extending circumferentially around the vessel by simply looking at a single projection.

FIG. 11 is a graphical image representation that embodies a technique that utilizes information calculated from IVUS imaging (or other imaging) and places a maximum thickness line 265 and a minimum thickness line 270 above and below the trace. Though not specific of where, circumferentially, the thickest portion of plaque occurs, the maximum thickness line 265 shows the exact maximum thickness of the plaque at each longitudinal position along the artery. In other words, a curving, continuous central axis parameter 275 follows the centerline of the artery and represents the axial location of the plaque, while a perpendicular axis parameter 280 represents the maximum thickness of the plaque by its distance from the edge of the vessel lumen trace 260. In a similar manner, the minimum thickness line 270 represents the minimum thickness of the plaque in the negative direction. It can be appreciated immediately while viewing the image/graphic combination depicted in FIG. 11 that the plaque is eccentric at various sections, even though there is no information present in this image/graphic combination to identify the exact circumferential angle where the maximum plaque thickness occurs. By viewing this image/graphic combination, the operator can immediately focus on the areas where the plaque is more eccentric, and the operator can also get a measurement of the minimum and maximum plaque thickness.

FIG. 12 illustratively depicts a graphical technique similar to that of FIG. 11, but with more specific information, namely the volume of plaque composition over a chosen length of vessel. A bar graph 285 is placed along-side the vessel lumen trace 260, and represents the volume of the different plaque components over a length of vessel. The user picks the proximal and distal point on the vessel which define a region of interest (for example a possible area of vulnerability), and the data obtained in this area is displayed with the bar graph 285. The bar graph 285 in this case represents four plaque components, fibrous 290, fibro-fatty 295, necrotic core 300, and dense calcium 305. The thickness (height in the radial direction) of each individual bar is proportional to the volume of that plaque component measured in a visually designated/indicated length of vessel. Each bar is color coded with a characteristic color to allow easier visual identification. For example, fibrous-dark green, fibrofatty-light green, necrotic core-red, dense calcium-white.

FIG. 13 illustratively depicts a graphical technique that is very similar to the one described in FIG. 11; however, instead of describing maximum and minimum plaque thickness at each axial location, the actual plaque thickness at each of the two sides is graphed. When the vessel lumen trace 260 is displayed in a two-dimensional mode, the upper thickness line 310 and the lower thickness line 315 graph the thickness of the plaque at points 180.degree. from each other (for example at twelve o'clock and six o'clock), depending on the orientation chosen for the vessel lumen trace 260.

The invention described herein is not limited to intravascular applications or even intraluminal applications. Tissue characterization is also possible in cancer diagnostics, and it is conceivable that a probe that images for cancer can also be used in conjunction with a three-dimensional map to create a similar reconstruction as that described above. This can be used to guide biopsy or removal techniques. Such cancers include, but are not limited to: prostate, ovarian, lung, colon and breast. In the intravascular applications, both arterial and venous imaging is conceived. Arteries of interest include, by way of example: coronaries, carotids, superficial femoral, common femoral, iliac, renal, cerebral and other peripheral and non-peripheral arteries.

The intravascular ultrasound methods described can also be expected to be applicable for other ultrasound applications, such as intracardiac echocardiography (ICE) or transesophageal echocardiography (TEE). Therapeutic techniques that are guided by these techniques include, but are not limited to, patent foramen ovale closure, atrial septal defect closure, ventricular septal defect closure, left atrial appendage occlusion, cardiac biopsy, valvuloplasty, percutaneous valve placement, trans-septal puncture, atrial fibrillation ablation (of pulmonary veins or left atrium, for example) and TIPS (transjugular intrahepatic portosystemic shunt for pulmonary hypertension).

Similar to the selective use of directional atherectomy and stenting/drugs in the circumferential, radial and axial orientations, the other energy delivery methods can also be manipulated as such. For example, in a thicker plaque, a higher power can be used in a cryogenic cooling catheter, etc. In addition, image guided automatic feedback can be used to automatically determine when to apply energy and when to stop applying energy, based on the information in the reconstruction. This is particularly of use in radiofrequency ablation of pulmonary veins for treatment of atrial fibrillation.

All of the image guided therapy described in this invention, can be conceived to be a combination of imaging and therapy on the same catheter, or to be two or more different catheters, each specialized in its use.

All of the techniques described here can also be used in conjunction with external imaging technologies such as MRI, CT, X-ray/angiography and ultrasound. Three dimensional reconstructions, for example from CT or MRI, can be co-registered with the imaging information in the same way as angiography.

The three-dimensional mapping of imaging information can also be combined with a three dimensional mapping of the electrical activity of the heart, for example, from information obtained from catheter-based electrodes. This is of use in a patient that has had an acute myocardial infarction.

It is also conceivable to include three-dimensional fluid mechanics analysis in the reconstruction so that points of high stress are identified.

Imaging Devices with Radiopaque Labels

Using the image collectors with radiopaque labels and the systems and method described herein, physicians and other users of intravascular imaging will be able to precisely locate the position of a given intravascular image within the vasculature. The inventions will speed intravascular imaging procedures, and result in less contrast and x-ray exposure for patients. The inventions will also make it easier for users to locate tissues of interest, e.g., thrombi, for accompanying endovascular procedures.

Any target can be imaged by methods and systems of the invention including, for example, bodily tissue. In certain embodiments, systems and methods of the invention image within a lumen of tissue. Various lumen of biological structures may be imaged including, but not limited to, blood vessels, vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, uterus and fallopian tubes, structures of the urinary tract including urinary collecting ducts, renal tubules, ureter, and bladder, and structures of the head and neck and pulmonary system including sinuses, parotid, trachea, bronchi, and lungs.

Any vascular imaging system may be used with the devices, systems, and methods of the invention including, for example, intravascular ultrasound (IVUS), intravascular Doppler, and intravascular optical coherence tomography (OCT). Devices, methods, and systems using the invention can also be used for intravascular visible imaging by co-locating a radiopaque label with a visible image collector, such as with an optical fiber or a CCD array camera. By co-locating a radiopaque label with the image collector, it is possible to track the location of the image collector, and thus, the image plane of the measurement. The radiopaque label will typically be quite small (1-5 mm) and constructed from a metal that does not transmit medical x-rays, such as platinum, palladium, rhenium, tungsten, tantalum, or combinations thereof.

### Catheters

When imaging vasculature, the imaging catheters are delivered to the tissue of interest via an introducer sheath placed in the radial, brachial or femoral artery. The introducer is inserted into the artery with a large needle, and after the needle is removed, the introducer provides access for guidewires, catheters, and other endovascular tools. An experienced cardiologist can perform a variety of procedures through the introducer by inserting tools such as balloon catheters, stents, or cauterization instruments. When the procedure is complete, the introducer is removed, and the wound can be secured with suture tape.

In certain embodiments, the invention provides systems and methods for imaging tissue using intravascular ultrasound (IVUS). IVUS uses a catheter with an ultrasound probe attached at the distal end. The proximal end of the catheter is attached to computerized ultrasound equipment. To visualize a vessel via IVUS, angiography is used while a technician/physician positions the tip of a guide wire. The physician steers the guide wire from outside the body, through angiography catheters and into the blood vessel branch to be imaged.

An exemplary IVUS catheter is shown in FIG. 21. Rotational imaging catheter 1000 is typically around 150 cm in total length can be used to image a variety of vasculature, such as coronary or carotid arteries and veins. When the rotational imaging catheter 1000 is used, it is inserted into an artery along a guidewire (not shown) to the desired location. Typically a portion of catheter, including a distal tip 1100, comprises a lumen (not shown) that mates with the guidewire, allowing the catheter to be deployed by pushing it along the guidewire to its destination.

An imaging assembly 1200 proximal to the distal tip 1100, includes transducers 1220 that image the tissue with ultrasound energy (e.g., 20-50 MHz range) and image collectors 1240 that collect the returned energy (echo) to create an intravascular image. The imaging assembly 1200 is shown in greater detail in FIG. 26.

As shown in FIG. 22, the imaging assembly 1200 comprises transducers 1220, image collectors 1240, radiopaque marker 1250, unibody 1260, and wiring bundle 1280. The imaging assembly 1200 is configured to rotate and travel longitudinally within imaging window 1300 allowing the imaging assembly 1200 to obtain 360° images of vasculature over the distance of travel. The imaging assembly is rotated and manipulated longitudinally by a drive cable (not shown) attached to inner member 1350. In some embodiments of rotational imaging catheter 1000, the imaging window can be over 15 cm long, and the imaging assembly 1200 can rotate and travel most of this distance, providing thousands of images along the travel. Because of this extended length of travel, it is especially useful to have radiopaque marker 1250 co-located with image collector 1240. That is, once the imaging assembly 1200 has been pulled back a substantial distance from the tip of the catheter, radiopaque marker 1250 allows a user to quickly verify the position of a given image rather than having to estimate with respect to the tip of the guidewire. In order to make locating an image easier, imaging window 1300 also has radiopaque markers 1370 spaced apart at 1 cm intervals.

Rotational imaging catheter 1000 additionally includes a hypotube 1400 connecting the imaging window 1300 and the imaging assembly 1200 to the ex-corporal portions of the catheter. The hypotube 1400 combines longitudinal stiffness with axial flexibility, thereby allowing a user to easily feed the catheter 1000 along a guidewire and around tortuous curves and branching within the vasculature. The ex-corporal portion of the hypotube includes shaft markers 1450 that indicate the maximum insertion lengths for the brachial or femoral arteries. The ex-corporal portion of catheter 1000 also include a transition shaft 1500 coupled to a coupling 1600 that defines the external telescope section 1650. The external telescope section 1650 corresponds to the pullback travel, which is on the order of 130 mm. The end of the telescope section is defined by the connector 1700 which allows the catheter 1000 to be interfaced to a patient interface module (PIM) which includes electrical connections to supply the power to the transducer and to receive images from the image collector. The connector 1700 also includes mechanical connections to rotate the imaging assembly 1200. When used clinically, pullback of the imaging assembly is also automated with a calibrated pullback device (not shown) which operates between coupling 1600 and connector 1700. Systems for IVUS are also discussed in U.S. Pat. No. 5,771,895; U.S. Pub. 2009/0284332; U.S. Pub. 2009/0195514 A1; U.S. Pub. 2007/0232933; and U.S. Pub. 2005/0249391, the contents of each of which are hereby incorporated by reference in their entirety.

The imaging assembly 1200 produces ultrasound energy and receives echoes from which real time ultrasound images of a thin section of the blood vessel are produced. The transducers 1220 are constructed from piezoelectric components that produce sound energy at 20-50 MHz. The image collector 1240 comprises separate piezoelectric elements that receive the ultrasound energy that is reflected from the vasculature. Alternative embodiments of imaging assembly 120 may use the same piezoelectric components to produce and receive the ultrasonic energy, for example, by using pulsed ultrasound. Another alternative embodiment may incorporate ultrasound absorbing materials and ultrasound lenses to increase signal to noise.

The imaging assembly 1200 used with the invention, including radiopaque marker 1250, is not limited to ultrasound applications, however. Radiopaque marker 1250 may be co-located with other image collectors, such as lenses, CCD arrays, and optical fibers, used with visible imaging, optical coherence tomography, or any other intravascular imaging system. Additionally, the radiopaque marker need not be disposed beneath, or interior to, the image collector. Alternative designs may have the radiopaque marker on top of, or external to, the image collector with windows or other openings that allow the image collector to function properly.

Regardless of the type of imaging, the radiopaque marker 1250 will be co-located longitudinally with respect to the image collector to allow a user to identify the location of the collector. Accordingly, radiopaque marker 1250 will be small in most instances, having a longitudinal dimension of less than 5 mm, e.g., less than 4 mm, e.g., less than 3 mm, e.g., less than 2 mm, e.g., less than 1 mm. The radiopaque marker 1250 will be at least 0.2 mm, e.g., at least 0.3 mm, e.g., at least 0.4 mm, e.g., at least 0.5 mm. The radiopaque marker 1250 may vary in axial size or diameter, depending upon its shape; however it will necessarily be small enough to fit within catheter 1000. For example radiopaque marker 1250 may have a diameter of at least 0.1 mm, e.g., at least 0.3 mm, e.g., at least 0.7 mm. The radiopaque marker 1250 may be constructed from any material that does not transmit x-rays and has suitable mechanical properties, including platinum, palladium, rhenium, tungsten, and tantalum.

Rotational imaging catheter 1000 can be used to obtain IVUS images such as shown in FIGS. 23-25. FIG. 23 (left hand side) shows an intravascular ultrasound image of a pulmonary artery, prior to placement of a stent. The border lines define the interior diameter of the lumen (blood vessel) and the shadow of the catheter. The shadow of the catheter serves as a calibration for luminal diameter. In other words, the ratio between the imaged area and the catheter shadow area can be used to calculate the actual luminal area at the point of imaging. However, while the absolute luminal area can be calculated from the intravascular image, the actual location of the luminal image is not evident from the intravascular image.

Accordingly, it is necessary to use a secondary imaging system, such as angiography, to determine the location of the image collector, and thus the acquired image. As discussed above, angiography uses a combination of x-ray imaging, typically fluoroscopy, and injected radiopaque contrasts to identify the structure of the vasculature. The real time image of the vasculature is typically displayed on a monitor during the intravascular procedure so that the technician or physician can watch the manipulation of the guidewire or catheter in real time. The angiogram may be processed with software and displayed on a computer, or the image may be a closed circuit image of a scintillating surface combined with a visibly fluorescent material. Newer fluoroscopes may use flat panel (array) detectors that are sensitive to lower doses of x-ray radiation and provide improved resolution over more traditional scintillating surfaces. An angiogram of a pulmonary artery is shown in the right hand image of FIG. 22.

### Imaging Systems

Using the devices of the invention, i.e., catheters with radiopaque labels co-located with the image collectors, improved systems for locating the position of an intravascular image can be provided. In principle, the methods can be as simple as imaging a portion of the vasculature of the subject using the image collector, e.g., as part of an imaging catheter, imaging the subject to determine the location of the radiopaque label co-located with an image collector, e.g., using angiography, and locating the position of the intravascular image, based upon the position of the radiopaque label.

A simple display using the described method is shown in FIG. 23, where the white box indicates the location of the left-hand intravascular image as defined by locating the radiopaque label (not shown in angiogram). In some embodiments, image tagging software can be used to automatically identify the location of the radiopaque label which will appear as a small spot having a darker color than the rest of the image. The image tagging software can automatically locate a box corresponding to the position of the image collector on the angiograph, e.g., as shown in FIG. 23. A physician using such this system will be able to locate specific structures of interest and return to those structures with less effort. Accordingly, the procedure will take less time, and the patient and the physician will be exposed to less x-ray radiation.

In addition to the embodiments described above, the devices, methods, and systems of the invention can be used to catalogue and display overlapping images of intravascular imaging and vascular structure, as is shown in FIGS. 24 and 25. Again, using image tagging software, or other algorithms, it is possible to display an angiogram that co-displays intravascular images. FIG. 24 shows a simulated IVUS image co-located with the location of the IVUS image on an angiogram of pulmonary arteries. FIG. 25 shows a simulated OCT image co-located with the location of the OCT image on an angiogram of pulmonary arteries. As discussed above, the principles of the invention using IVUS or OCT are identical once the radiopaque label has been co-located with the image collector.

In other embodiments, an angiogram, or more likely a simulated angiogram, can be used after the procedure to post-operatively examine the vasculature of the patient. Using the images of FIGS. 24 and 25, a technician or physician can later scroll over the angiogram and click on specific vasculature to examine the corresponding intravascular image. Accordingly, the methods and systems of the invention can provide a more complete picture of the cardiovascular health of the patient. Further improvements on the system could use automatic border detection and/or color labeling as described in U.S. Patent Publication No. 2008/0287795, incorporated herein by reference in its entirety.

A flowchart 2000 of a system of the invention is shown in FIG. 26. At step 2100 intravascular imaging data, such as from an imaging catheter having a radiopaque label co-located with the image collector, is received. At step 2200 vasculature imaging data, such as from a fluoroscope, is received. At step 2300 the vasculature imaging data is analyzed to determine if the radiopaque label is identifiable. If the label is not identifiable, the system receives new vasculature imaging data. If the label is identifiable, the system proceeds to output a vascular image, such as an angiogram, showing the location of the intravascular image. Then the system also outputs the intravascular image, e.g., an IVUS or OCT image. In some embodiments, the system simultaneously outputs both the angiogram and intravascular image in the same image (dashed box).

A system of the invention may be implemented in a number of formats. An embodiment of a system 3000 of the invention is shown in FIG. 27. The core of the system 3000 is a computer 3600 or other computational arrangement (see FIG. 28) comprising a processor 3650 and memory 3670. The memory has instructions which when executed cause the processor to receive imaging data of vasculature of a subject collected with an image collector co-located with a radiopaque label. The imaging data of vasculature will typically originate from an intravascular imaging device 3200, which is in electronic and/or mechanical communication with an imaging catheter 3250. The memory additionally has instructions which when executed cause the processor to receive an image of the subject including the radiopaque label. The image of the subject will typically be an x-ray image, such as produced during an angiogram or CT scan. The image of the subject will typically originate in an x-ray imaging device 3400, which is in electronic and/or mechanical communication with an x-ray source 3430 and an x-ray image collector 3470 such as a flat panel detector, discussed above. Having collected the images, the processor then processes the image, and outputs an image of the subject showing the location of the image collector, as well as an image of the vasculature of a subject. The images are typically output to a display 3800 to be viewed by a physician or technician. In some embodiments a displayed image will simultaneously include both the intravascular image and the image of the vasculature, for example as shown in FIGS. 24 and 25.

In advanced embodiments, system 3000 may comprise an imaging engine 3700 which has advanced image processing features, such as image tagging, that allow the system 3000 to more efficiently process and display combined intravascular and angiographic images. The imaging engine 3700 may automatically highlight or otherwise denote areas of interest in the vasculature. The imaging engine 3700 may also produce 3D renderings of the intravascular images and or angiographic images. In some embodiments, the imaging engine 3700 may additionally include data acquisition functionalities (DAQ) 3750, which allow the imaging engine 3700 to receive the imaging data directly from the catheter 3250 or collector 3470 to be processed into images for display.

Other advanced embodiments use the I/O functionalities 3620 of computer 3600 to control the intravascular imaging 3200 or the x-ray imaging 3400. In these embodiments, computer 3600 may cause the imaging assembly of catheter 3250 to travel to a specific location, e.g., if the catheter 3250 is a pull-back type. The computer 3600 may also cause source 3430 to irradiate the field to obtain a refreshed image of the vasculature, or to clear collector 3470 of the most recent image. While not shown here, it is also possible that computer 3600 may control a manipulator, e.g., a robotic manipulator, connected to catheter 3250 to improve the placement of the catheter 3250.

A system 4000 of the invention may also be implemented across a number of independent platforms which communicate via a network 4090, as shown in FIG. 28. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

As shown in FIG. 28, the intravascular imaging system 3200 and the x-ray imaging system 3400 are key for obtaining the data, however the actual implementation of the steps, for example the steps of FIG. 26, can be performed by multiple processors working in communication via the network 4090, for example a local area network, a wireless network, or the internet. The components of system 4000 may also be physically separated. For example, terminal 4670 and display 3800 may not be geographically located with the intravascular imaging system 3200 and the x-ray imaging system 3400.

As shown in FIG. 28, imaging engine 8590 communicates with host workstation 4330 as well as optionally server 4130 over network 4090. In some embodiments, an operator uses host workstation 4330, computer 4490, or terminal 4670 to control system 4000 or to receive images. An image may be displayed using an I/O 4540, 4370, or 4710, which may include a monitor. Any I/O may include a monitor, keyboard, mouse or touch screen to communicate with any of processor 4210, 4590, 4410, or 4750, for example, to cause data to be stored in any tangible, nontransitory memory 4630, 4450, 4790, or 4290. Server 4130 generally includes an interface module 4250 to communicate over network 4090 or write data to data file 4170. Input from a user is received by a processor in an electronic device such as, for example, host workstation 4330, server 4130, or computer 4490. In certain embodiments, host workstation 4330 and imaging engine 8550 are included in a bedside console unit to operate system 4000.

In some embodiments, the system may render three dimensional imaging of the vasculature or the intravascular images. An electronic apparatus within the system (e.g., PC, dedicated hardware, or firmware) such as the host workstation 4330 stores the three dimensional image in a tangible, non-transitory memory and renders an image of the 3D tissues on the display 3800. In some embodiments, the 3D images will be coded for faster viewing. In certain embodiments, systems of the invention render a GUI with elements or controls to allow an operator to interact with three dimensional data set as a three dimensional view. For example, an operator may cause a video affect to be viewed in, for example, a tomographic view, creating a visual effect of travelling through a lumen of vessel (i.e., a dynamic progress view). In other embodiments an operator may select points from within one of the images or the three dimensional data set by choosing start and stop points while a dynamic progress view is displayed in display. In other embodiments, a user may cause an imaging catheter to be relocated to a new position in the body by interacting with the image.

In some embodiments, a user interacts with a visual interface and puts in parameters or makes a selection. Input from a user (e.g., parameters or a selection) are received by a processor in an electronic device such as, for example, host workstation 4330, server 4130, or computer 4490. The selection can be rendered into a visible display. In some embodiments, an operator uses host workstation 4330, computer 4490, or terminal 4670 to control system 4000 or to receive images. An image may be displayed using an I/O 4540, 4370, or 4710, which may include a monitor. Any I/O may include a keyboard, mouse or touch screen to communicate with any of processor 4210, 4590, 4410, or 4750, for example, to cause data to be stored in any tangible, nontransitory memory 4630, 4450, 4790, or 4290. Server 4130 generally includes an interface module 4250 to effectuate communication over network 4090 or write data to data file 4170. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections). In certain embodiments, host workstation 4330 and imaging engine 8550 are included in a bedside console unit to operate system 4000.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, NAND-based flash memory, solid state drive (SSD), and other flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server 4130), a middleware component (e.g., an application server), or a front-end component (e.g., a client computer 4490 having a graphical user interface 4540 or a web browser through which a user can interact with an implementation of the subject matter described herein), or any combination of such back-end, middleware, and front-end components. The components of the system can be interconnected through network 4090 by any form or medium of digital data communication, e.g., a communication network. Examples of communication networks include cell networks (3G, 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include programming language known in the art, including, without limitation, C, C++, Perl, Java, ActiveX, HTML5, Visual Basic, or JavaScript.

A computer program does not necessarily correspond to a file. A program can be stored in a portion of file 4170 that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 4090 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment) into patterns of magnetization by read/write heads, the patterns then representing new collocations of information desired by, and useful to, the user. In some embodiments, writing involves a physical transformation of material in tangible, non-transitory computer readable media with certain properties so that optical read/write devices can then read the new and useful collocation of information (e.g., burning a CD-ROM). In some embodiments, writing a file includes using flash memory such as NAND flash memory and storing information in an array of memory cells include floating-gate transistors. Methods of writing a file are well-known in the art and, for example, can be invoked automatically by a program or by a save command from software or a write command from a programming language.

In certain embodiments, display 3800 is rendered within a computer operating system environment, such as Windows, Mac OS, or Linux or within a display or GUI of a specialized system. Display 3800 can include any standard controls associated with a display (e.g., within a windowing environment) including minimize and close buttons, scroll bars, menus, and window resizing controls. Elements of display 3800 can be provided by an operating system, windows environment, application programming interface (API), web browser, program, or combination thereof (for example, in some embodiments a computer includes an operating system in which an independent program such as a web browser runs and the independent program supplies one or more of an API to render elements of a GUI). Display 380 can further include any controls or information related to viewing images (e.g., zoom, color controls, brightness/contrast) or handling files comprising three-dimensional image data (e.g., open, save, close, select, cut, delete, etc.). Further, display 3800 can include controls (e.g., buttons, sliders, tabs, switches) related to operating a three dimensional image capture system (e.g., go, stop, pause, power up, power down).

In certain embodiments, display 3800 includes controls related to three dimensional imaging systems that are operable with different imaging modalities. For example, display 3800 may include start, stop, zoom, save, etc., buttons, and be rendered by a computer program that interoperates with IVUS, OCT, or angiogram modalities. Thus display 380 can display an image derived from a three-dimensional data set with or without regard to the imaging mode of the system.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for constructing an image of a body structure, the method comprising:
externally imaging a body structure within the patient using a first imaging device;
internally imaging the body structure within the patient using a second imaging device that comprises a radiopaque label co-located with an image collector of the second imaging device; and
combining external imaging data and internal imaging data to produce an image of the body structure, wherein the label on the second imaging device facilitates alignment of the external imaging data and the internal imaging data.

2. The method according to claim 1, wherein the first imaging device is capable of detecting the label on the second imaging device.

3. The method according to claim 1, wherein the first imaging device is an angiography system.

4. The method according to claim 1, wherein the body structure is a vessel.

5. The method according to claim 4, wherein the vessel is part of the patient's cardiovascular system.

6. The method according to claim 1, wherein the image collector is a piezoelectric sensor, a micromachined transducer, a photodiode, a charge coupled device, a microchannel array, a lens, or an optical fiber.

7. The method according to claim 1, wherein the radiopaque label is less than 3 mm in length measured longitudinally along the catheter.

8. The method according to claim 1, wherein the radiopaque label comprises platinum, palladium, rhenium, tungsten, or tantalum.

9. The method according to claim 1, wherein the image collector is capable of being translated while imaging vasculature.

10. The method according to claim 9, wherein the image collector is capable of being translated proximally while imaging vasculature.

11. The method according to claim 1, wherein the imaging collector is capable of collecting intravascular ultrasound imaging data.

12. The method according to claim 1, wherein the imaging collector is capable of collecting intravascular optical coherence tomography imaging data.

13. The method according to claim 1, further comprising displaying an image of the subject including the radiopaque label.

14. A system for constructing an image of a body structure, comprising:
a processor; and
a computer readable storage medium having instructions that when executed cause the processor to:
receive a first set of imaging data of a body structure of a patient acquired from a first imaging device that is external to the patient;
receive a second set of imaging data of a body structure of a patient acquired from an image collector of a second imaging device from inside the patient, the second data set comprising a radiopaque label within the data;
use the radiopaque label to facilitate aligning the first set of imaging data and the second set of imaging data; and
output and image of the body structure.

15. The system according to claim 14, wherein the image comprises the radiopaque label.

16. The system according to claim 14, wherein the image collector is a piezoelectric sensor, a micromachined transducer, a photodiode, a charge coupled device, a microchannel array, a lens, or an optical fiber.

17. The system according to claim 14, wherein the first imaging device is an angiography system.

18. The system according to claim 14, wherein the body structure is a vessel.

19. The system according to claim 14, wherein the vessel is part of the patient's cardiovascular system.
